(19) United States
(12) Patent Application Publication
(10) Pub. No.: US 2014/0276848 A1
(43) Pub. Date: Sep. 18, 2014

(54) TISSUE MODIFICATION DEVICES

(71) Applicants: Roy LEGUIDLEGUID, Union City, CA (US); Michael P. WALLACE, Pleasanton, CA (US); Nestor C. CANTORNA, Union City, CA (US)

(72) Inventors: Roy LEGUIDLEGUID, Union City, CA (US); Michael P. WALLACE, Pleasanton, CA (US); Nestor C. CANTORNA, Union City, CA (US)

(21) Appl. No.: 14/209,418

(22) Filed: Mar. 13, 2014

Related U.S. Application Data
(60) Provisional application No. 61/780,421, filed on Mar. 13, 2013.

(51) Int. Cl. A61B 17/16

(52) U.S. Cl. CPC A61B 17/1604; A61B 17/1671
USPC 606/83

(57) ABSTRACT

Tissue modification devices, and particularly very low-profile, yet strong and manipulatable Rongeur devices. In general, these devices are low-profile, with a flat and thin distal end that may be much thinner than it is wide. The distal end includes a cutting window within which one or more blades move to cut tissue. The distal end region and the curved region forming an angle with an elongate rigid body may be sufficiently stiff and rigid so that the distal end can be pushed (or pulled) against a tissue to be cut with sufficient force so that even hard tissue such as bone may be held within the window and cut by the blade(s).

### CROSS REFERENCE TO RELATED APPLICATIONS

This patent application claims priority to U.S. provisional patent application No. 61/780,421, filed on Mar. 13, 2013, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates generally to medical/surgical devices and methods. More specifically, the present invention relates to thin, curved tissue modification devices and methods of modifying tissue using such devices, particularly for treatment of spinal stenosis.

### BACKGROUND

A significant number of surgical procedures involve modifying tissue in a patient's body, such as by removing, cutting, shaving, abrading, shrinking, ablating or otherwise modifying tissue. Minimally invasive (or “less invasive”) surgical procedures often involve modifying tissue through one or more small incisions or percutaneous access, and thus may be more technically challenging procedures. Some of the challenges of minimally invasive tissue modification procedures include working in a smaller operating field, working with, smaller devices, and trying to operate with reduced or even no direct visualization of the tissue (or tissues) being modified. For example, using arthroscopic surgical techniques for repairing joints such as the knee or the shoulder, it may be quite challenging to modify certain tissues to achieve a desired result, due to the required small size of arthroscopic instruments, the confined surgical space of the joint, lack of direct visualization of the surgical space, and the like. It may be particularly challenging in some surgical procedures, for example, to cut or contour bone or ligamentous tissue with currently available minimally invasive tools and techniques. For example, trying to shave a thin slice of bone off a curved bony surface, using a. small-diameter tool in a confined space with little or no ability to see the surface being cut, as may be required in some procedures, may be incredibly challenging or even impossible using currently available devices.

One area of surgery which would likely benefit from the development of less invasive techniques is the treatment of spinal stenosis. Spinal stenosis occurs when nerve tissue and/or the blood vessels supplying nerve tissue in the spine become impinged by one or more structures pressing against them, causing symptoms. The most common form of spinal stenosis occurs in the lower (or lumbar) spine and can cause severe pain, numbness and/or loss of function in the lower back and/or one or both lower limb.

FIG. 1 is a top view of a vertebra with the cauda equina (the bundle of nerves that extends from the base of the spinal cord) shown in cross section and two nerve roots branching from the cauda equina to exit the central spinal canal and extend through intervertebral foramina on either side of the vertebra. Spinal stenosis can occur when the spinal cord, cauda equina and/or nerve root(s) are impinged by one or more tissues in the spine, such as buckled or thickened ligamentum flavum, hypertrophied facet joint (shown as superior articular processes in FIG. 1), osteophytes (or “bone spurs”) on vertebrae, spondylolisthesis (sliding of one vertebra relative to an adjacent vertebra), facet joint synovial cysts, and/or collapse, bulging or herniation of an intervertebral disc. Impingement of neural, and/or neurovascular tissue in the spine by one or more of these tissues may cause pain, numbness and/or loss of strength or mobility in one or both of a patient's lower limbs and/or of the patient's back.

In the United States, spinal stenosis occurs with an incidence of between 4% and 6% (or more) of adults aged 50 and older and is the most frequent reason cited for back surgery in patients aged 60 and older. Patients suffering from spinal stenosis are typically first treated with conservative approaches such as exercise therapy, analgesics, anti-inflammatory medications, and epidural steroid injections. When these conservative treatment options fail and symptoms are severe, as is frequently the case, surgery may be required to remove impinging tissue and decompress the impinged nerve tissue.

Lumbar spinal stenosis surgery involves first making an incision in the back and stripping muscles and supporting structures away from the spine to expose the posterior aspect of the vertebral column, Thickened ligamentum flavum is then exposed by complete or partial removal of the bony arch (lamina) covering the back of the spinal, canal (laminectomy or laminotomy). In addition, the surgery often includes partial or complete facetectomy (removal of all or part of one or more facet joints), to remove impinging ligamentum flavum or bone tissue. Spinal stenosis surgery is performed under general anesthesia, and patients are usually admitted to the hospital for five to seven days after surgery, with full recovery from surgery requiring between six weeks and three months. Many patients need extended therapy at a rehabilitation facility to regain enough mobility to live independently.

Removal of vertebral bone, as occurs in laminectomy and facetectomy, often leaves the affected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments, Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal, anatomy, and increase long-term morbidity above levels seen in untreated patients.

Therefore, it would be desirable to have less invasive methods and devices for modifying target tissue in a spine to help ameliorate or treat spinal stenosis, while inhibiting unwanted damage to non-target tissues. Ideally, such techniques and devices would reduce neural and/or neurovascular impingement without removing significant amounts of vertebral bone, joint, or other spinal support structures, thereby avoiding the need for spinal fusion and, ideally, reducing the long-term morbidity resulting from currently available surgical treatments. It may also be advantageous to have minimally invasive or less invasive tissue modification devices capable of treating target tissues in parts of the body other than the spine. At least some of these objectives will be met by the present invention.

### SUMMARY OF THE DISCLOSURE

Described herein are tissue modification devices, and in particular, very low-profile and yet strong Rongeur devices. In general, the devices have a flat and thin distal region, which may be curved and/or tapered, and the distal region is typically much thinner than it is wide. The distal end region may include a cutting window. One or more blades may move along the window to cut tissue. Despite begin very thin (e.g. between 0.5-5 mm at maximum thickness) the distal end region may be sufficiently stiff and rigid so that it can be pushed (or pulled) against a tissue to be cut with sufficient force so that the one or more blades (which may be proud of the distal end region) may cut even tissues such as bone or other calcified regions. The blades may move proximally to distally within the cut window, and may extend above the distal end region. The overall rigidity and stiffness of the devices, and particularly the distal end region, may be enhanced by the bend region that forms the angle between the proximal elongate body of the device and the end region. For example, the bend region may include a longitudinal crease, or invagination which enhances stiffness and resists bending. In some variations, the bend region has a cross-section that is I-beam or C-beam like, to enhance strength and resist bending. The bend region may form an angle of between about 90 degrees and 180 degrees (e.g. no bend) between the distal end region and the proximal elongate region.

For example, described herein are devices for modifying tissue. A device for modifying tissue (including, in particular, spinal tissues) may include: a rigid elongate body having a thin distal portion that is bent relative to the elongate body, wherein the distal portion comprises a cutting window and the distal portion is slightly wider than the elongate body; a bent region between the elongate body and the distal portion, wherein the bent region is rigid; a movable blade disposed along one side of the cutting window and configured to extend across the cutting window to cut tissue; and a handle at the proximal end of the body, wherein the handle includes an actuator configured to move the movable blade towards a second blade; wherein the distal portion has a height, a length and a width, and wherein the height is less than the width, further wherein the height is not more than 10 mm at any point along its length, and the width is not more than 20 mm at any point along its length.

As mentioned, the bent region may include an invagination (e.g., a longitudinal, proximal-to-distal fold or invagination) that is configured to stiffen the bent region.

In general, the distal end region (the distal portion) is thin. For example, the distal portion may have a height of not more than 5 mm at any point along its length and a width of not more than 10 mm at any point along its length. The distal portion may have a height of not more than 2 mm at any point along its length and a width of not more than 5 mm at any point along its length. The height of the distal portion may exclude the cutting members, e.g., the movable blade and the second blade, which may slide within the cutting window and may extend above the height of the distal portion. In some variations the cutting member extends between 0.5 mm and 5 mm above the height of the distal portion. In some variations the cutting member(s) is/are flush with the height of the distal portion. In some variations the cutting member(s) is/are retractable within the distal portion, so that in a first position they do not extend above the height of the distal portion but in a second position they do.

In any of the variations described, the second blade may be configured to move across the cutting window towards the movable blade when the movable blade extends across the cutting window. Thus the first (movable blade) and the second blade both move towards each other to cut tissue within the cutting window.

The first (e.g., movable) blade and the second movable blade may be adapted to cut tissue by including a sharp cutting edge; the cutting edge may be serrated and/or angled, and/or ridged, etc.

Either or both the moveable blade and the second blade may be actuated my a tendon, pull wire, push rod, or the like. For example, the actuator may be configured to pull the second blade toward the first blade. Thus, the device may include a pull-wire configured to pull the movable blade towards the second blade, and/or a pull-wire configured to pull the second blade towards the movable blade, and/or a pull-wire configured to pull the movable blades towards each other. Alternatively or additional, the actuator may be configured to push the first blade toward the second blade. The blades may also include a restoring pull-wire or pushing mechanism. The actuator may include a bias (e.g., spring, etc.) or return to restore the blade to a position or to assist in displacing the blade toward the second blade (or towards each other), which may decrease the effort on the user to cut the tissue.

In some variations the distal portion of the device is curved (e.g., crescent-shaped), or may be flat.

Any of the devices described herein may also include a guidewire coupler and/or guidewire channel for interaction with a guidewire. In some variations the guidewire is adapted to be attached to the distal end of the device (e.g., fixedly coupled, e.g., end-to-end with a guidewire); in other variations the device includes a lumen or channel extending along all or a portion of the distal end of the device in which a guidewire may pass, so that the distal end of the device may be slide along a guidewire.

For example, a device for modifying tissue may include: a rigid elongate body having a thin distal portion that is bent at a fixed angle between about 180 and 90 degrees relative to the elongate body, wherein the distal portion comprises a cutting window, and wherein the distal portion is slightly wider than the elongate body; a rigid bent region forming the fixed angle between the elongate body and the distal portion; a movable blade disposed along one side of the cutting window and configured to extend across the cutting window to cut tissue; and a handle at the proximal end of the body, wherein the handle includes an actuator configured to move the movable blade towards a second blade; wherein the distal portion has a height, a length and a width, and wherein the height is less than the width, further wherein the height is not more than 5 mm at any point along its length, and the width is not more than 10 mm at any point along its length.

In another example, a device for modifying tissue includes: a rigid elongate body having a thin distal portion that is bent at a fixed angle between about 180 and 90 degrees relative to the elongate body, wherein the distal portion comprises a cutting window, and wherein the distal portion is slightly wider than the elongate body; a rigid, invaginated, bent region forming the fixed angle between the elongate body and the distal portion; a movable blade disposed along one side of the cutting window and configured to extend across the cutting window to cut tissue; and a handle at the proximal end of the body, wherein the handle includes an actuator configured to move the movable blade towards a second blade; wherein the distal portion has a height, a length and a width, and wherein the height is less than the width, further wherein the height is not more than 5 mm at any point along its length, and the width is not more than 10 mm at any point along its length.

### BRIEF DESCRIPTION OF THE DRAWINGS

FIG. 1 is a top view of a vertebra with the cauda equina shown in cross section and two nerve roots branching from the cauda equina to exit the central spinal canal and extend through intervertebral foramina on either side of the vertebra.

FIG. 2 is a top view of a vertebra shown in cross section with a conventional Rongeur,

FIG. 3 is a top view of a vertebra shown in cross section showing the central canal, lateral recess, and foraminal regions.

FIGS. 4, 5A, and 5B illustrate a particular example of target tissue that may be removed with the devices and methods described herein.

FIGS. 6A, 6B and 6C show top perspective, side and alternate side view comparisons of a prior art surgical Rongeur and the thin and rigid surgical Rongeurs described herein.

FIG. 7 shows an example of a thin Rongeur apparatus as described, including a handle.

FIGS. 8A, and 8B show the distal end, including a portion of an elongate body, rigid curved region and distal portion including a window having a movable blade that slides longitudinally within the window to cut tissue.

FIGS. 9A and 9B show two variations of Rongeur-type tissue modification apparatuses, including the rigid (and longitudinally invaginated) curved region and a distal portion including a cutting window.

FIG. 10A shows one variation of a distal end region of a curved tissue modification devices for removing impinging tissue including a guidewire coupling region.

FIG. 10B shows another variation of a distal end region of a curved tissue modification device.

### DETAILED DESCRIPTION

Various embodiments of tissue modification devices and systems, as well as methods for making and using tissue modification devices and systems, are provided herein. In general, a curved tissue-modification device as described herein is configured to remove tissue from a patient. In particular, these tissue-modification devices may be configured to decompress spinal stenosis. These devices typically include a curved elongate body that extends proximally to distally (proximal/distal), and is configured to be inserted into a patient so that it extends around the target tissue, so that it can be driven against the target tissue. Thus, the device may be extended into, through, and/or around a spinal foramen. For example, in variations in which the device has an elongated shape that is long and flat with a width greater than the thickness, the device includes a first major surface (e.g., a front) and a second major surface (a back), and has edges (minor surfaces) between the first and second major surfaces. The first major surface may be referred to as the anterior or front surface and the second major surface may be referred to as the posterior or back surface. The devices described herein may be flexible along the anterior and posterior surfaces, and the anterior or front surface may include one or more cutting edges configured to cut tissue as the anterior surface of the device is urged against a tissue. The posterior surface may be configured to shield or protect non-target tissue.

Although much of the following description and accompanying figures generally focuses on surgical procedures in spine, in alternative embodiments, devices, systems and methods of the present invention may be used in any of a number of other anatomical locations in a patient's body. For example, in some embodiments, the tissue modification devices of the present invention may be used in minimally invasive procedures in the shoulder, elbow, wrist, hand, hip, knee, foot, ankle, other joints, or other anatomical locations in the body. Similarly, although some embodiments may be used to remove or otherwise modify ligamentum flavum and/or bone in a spine to treat spinal stenosis, in alternative embodiments, other tissues may be modified to treat any of a number of other conditions. For example, in various embodiments, treated tissues may include but are not limited to ligament, tendon, bone, tumor, cyst, cartilage, scar, osteophyte, inflammatory tissue and the like. Non-target tissues may include neural tissue and/or neurovascular tissue in some embodiments or any of a number of other tissues and/or structures in other embodiments. In one alternative embodiment, for example, a flexible tissue modification device may be used to incise a transverse carpal ligament in a wrist while inhibiting damage to the median nerve, to perform a minimally invasive carpal tunnel release procedure. Thus, various embodiments described herein may be used to modify any of a number of different tissues, in any of a number of anatomical locations in the body, to treat any of a number of different conditions.

FIG. 2 is a top view of a vertebra with the cauda equina (the bundle of nerves that extends from the base of the spinal cord) shown in cross section and two nerve roots branching from the cauda equina to exit the central spinal canal and extend through, intervertebral foramina on either side of the vertebra. Spinal stenosis can occur when the spinal cord, cauda equine and/or nerve root(s) are impinged by one or more tissues in the spine, such as buckled or thickened ligamentum flavum, hypertrophied facet joint, osteophytes (or “bone spurs”) on vertebrae, spondylolisthesis (sliding of one vertebra relative to an adjacent vertebra), facet joint. synovial cysts, and/or collapse, bulging or herniation of an intervertebral disc, As shown in FIG. 2, the tissue may be the impinging nerve and/or vascular tissue. Impingement of neural and/or neurovascular tissue in the spine by one or more of these tissues may cause pain, numbness and/or loss of strength or mobility in one or both of a patient's lower limbs and/or of the patient's back.

As described above, conventional lumbar spinal stenosis surgery involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or lamitiotomy). As shown, conventional large, straight, rigid tools, such as Rongeurs or bone punches are brought into the spine to attempt to remove the impinging tissue. As shown, due to their size and shape, conventionally tools, and particularly conventional Rongeurs, are unable to access the impinging tissue in the lateral recess and foraminal regions (as shown in FIG. 3) and are therefore not able to adequately reach the tissue to perform a complete decompression. In an attempt to remove more of the impinging tissue, the surgery often includes partial or complete facetectomy (removal of all. or part of one or more facet joints), to remove impinging ligamentum flavum or bone tissue.

FIG. 3 is a top view of a vertebra shown in cross section showing the central canal, lateral recess, and foraminal regions. Patients suffering from lumbar spinal stenosis may have impinging tissue in just the central canal, the lateral recess, or the foraminal region of one or more vertebra. Alternatively, a patient may have impinging tissue in a combination of regions, for example central with lateral recess stenosis, lateral recess with foraminal stenosis, all three regions or any other combination. Conventional tools are unable to remove tissue from the lateral recess and especially the foraminal region. Furthermore, if a patient has the majority of their stenosis in the lateral recess, it may be desirable to decompress only the lateral recess rather than decompressing the central canal and/or the foraminal region. As shown in FIG. 4, the devices described herein may remove tissue from the lateral recess while not significantly removing tissue far out in the foraminal region. FIGS. 6 through 10B illustrate an improved device having an elongate body sized and configured so that it does not extend far out into the foramen. The devices described herein may be useful for decompressing foraminal stenosis as well. FIG. 4 is an anterior view of two vertebrae. The two pedicles of each vertebra are highlighted. As shown, device 400 has been places through an interlaminar window, for example, and advanced from the central canal toward the foramen (between two pedicles), such that the lateral, recess can be decompressed; ligamentus tissue and some bony tissue in the lateral recess may be removed with device 400. In FIGS. 5A and 5B, device 500 may be advanced through an interlaminar window and around a facet joint. Once in position, the at least partially rigid device may be pulled up against, the soft and bony tissue, within the lateral recess for example, to capture and remove tissue as described below.

Removal of vertebral bone, as occurs in laminectomy and facetectomy, often leaves the affected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

Described herein are tissue modification devices and methods for removing target impinging tissue while sparing healthy tissue. These devices may be referred to as biting Rongeurs. FIGS. 6A through 10 illustrate exemplary curved tissue modification devices (Rongeurs) for removing impinging tissue. As shown in FIG. 6A, a curved, thin profile tissue modification device 600 is compared to a conventional surgical Rongeur 601. As shown, the device 600 includes an elongate body 602, having an axial length, a width and a thickness, wherein the axial length is greater than the width and the width is greater than the thickness. As shown, the thickness of the device 600 is substantially thinner than the conventional Rongeur 601. The Rongeur 601 may have a thickness or height between 4 and 10 mm. In one specific example, as shown in FIG. 6C, the Rongeur may have a height of about 6 mm. As shown, the device 600 may have a height of only about 2 mm. In some embodiments the device thickness may be between 0.1 mm and 4 mm. In some embodiments, the width of the elongate body 602 of device 600 may be between 1 and 15 mm. In some embodiments, the width of the elongate body may be between 2 and 8 mm, while in some embodiments, the width may be between 3 and 5 mm. In one particular example, the width of the elongate body may be about 4 mm. The distal end region (distal portion) of the device 600 is flat and wider than the more proximal elongate body (shaft). The rigid bend region 605 may be flanged out. The bend region 605 may also include a crease (e.g., invagination) that extends longitudinally. This invagination may support the bend and provide additional stiffness to this region, allowing the distal region to be forced against tissue so that target tissue to be cut can be held within the cutting window even when it may resist cutting. FIG. 9B also shows a creased region 909.

As shown in FIG. 7, the device 700 may include an elongate body 702, a handle 704 with an actuator 706, one or more tissue modifying members 708 and 710, and one or more protective surfaces 712. In some embodiments, the elongate body may further include a rigid shaft that couples the distal portion of the elongate body to the handle. In some embodiments, as shown in FIG. 7, a distal portion of the device is curved such that there is an angle between the rigid shaft and the distal portion of the elongate body. In some embodiments, the angle is between 180 degrees and 90 degrees, while in some embodiments, the angle is less than 90 degrees. In the embodiment shown, the tissue modifying members comprise blades, although in alternative embodiments other tissue modifying members may be added or substituted. Tissue modification via tissue modifying members may include cutting, ablating, dissecting, repairing, reducing blood flow in. shrinking, shaving, burring, biting, remodeling, biopsying, debriding, lysing, debulking, sanding, filing, planing, heating, cooling, vaporizing, delivering a drug to, and/or retracting the target tissue. In some embodiments (e.g., FIG. 10A), the device may further include a guidewire coupler 933 at the distal end. The guide wire coupler may be configured to couple to a guidewire such that a guidewire may be removably coupled to the distal end of the device. The guidewire may be used to position and/or apply a distal tensioning force to the device to aid in tissue capture and removal.

In various embodiments, elongate body 702 may have any number of dimensions, shapes, profiles and amounts of flexibility or rigidity. In various embodiments, elongate body 108 may have one or more of a round, ovoid, ellipsoid, flat, cambered flat, rectangular, square, triangular, symmetric or asymmetric cross-sectional shape. As shown in FIGS. 8A and 8B, in the pictured embodiment, elongate body 702 may have a relatively flat configuration, which may facilitate placement of body 702 between target and non-target tissues. Distal portion of body 702 may be thin and/or tapered, to facilitate its passage into or through narrow spaces as well as through small incisions on a patient's skin. Body 702 may also include a slightly widened portion around the area of window 714 and blades. In some embodiments, the window or portion of device between the blades may be curved. Alternatively, as shown in FIGS. 8A and 8B, the window or portion of device between the blades may be straight. In one embodiment, such as an embodiment used for modifying tissue in a spine, body 702 may have a small profile, e.g., having a height of not more than 10 mm at any point along its length and a width of not more than 20 mm at any point along its length, or more preferably a height not more than 5 mm at any point along its length and a width of not more than 10 mm at any point along its length, or even more preferably a height not more than 2 mm at any point along its length and a width of not more than 4 mm at any point along its length. Body 702 may be long enough to extend through a first incision on a patient, between target and non-target tissue, and out a second incision on a patient. Alternatively, body 702 may be long enough to extend through a first incision, between the target and non-target tissue, and to an anchoring location within the patient In another alternative embodiment, body 702 may be long enough to extend through a first incision, between the target and non-target tissue, to a location nearby but distal to the target tissue within the patient, with some portion of tissue modification device 700 anchored to a guidewire (as described above, not shown). In some embodiments, elongate body 702 includes at least one feature for allowing passage of the body over a guidewire or other guide member or to allow passage of one or more guide members over or through body 702. For example, in various embodiments, body 702 may include one or more guidewire lumens, rails, tracks, lengthwise impressions or some combination thereof.

In some embodiments, it may be advantageous to include one or more rigid sections in elongate body 702, such as to impart pushability to a portion of body 702 or to facilitate application of force to tissue modification members 708 and 710 without causing unwanted bending or kinking of elongate body 702. In such embodiments, rigidity may be conferred by using additional materials in body 702 or by making the rigid portions thicker or wider or of a different shape.

Handle 704 may have any suitable configuration according to various embodiments. Similarly, actuator 706 may include any of a number of actuation devices in various embodiments. In the embodiment shown in FIG. 7, actuator 706 comprises a trigger or moving handle portion, which is grasped by a user and pulled or squeezed toward handle 704 to bring blades 708 and 710 together to cut tissue. In an alternative embodiment, actuator 706 instead may include a switch or button for activating a radiofrequency surgical ablation tissue modifying member. In yet another embodiment, actuator 106 may include a combination trigger and switch, one or more pull wires, any suitable form of lever and/or some combination thereof.

FIGS. 8A and 8B show in greater detail a portion of tissue modification device 700. In these figures, window 714 and blades 708 and 710 are more clearly seen. In one embodiment, as shown, at least a portion of elongate body and blades may have a slightly curved configuration. In alternative embodiments, at least a portion of elongate body and blades may be flat. In other alternative embodiments, tissue modification members such as blades may be proud to the elongate body.

Blades 708 and 71.0 include a distal 708 and a proximal blade 710 that reside at the distal and proximal edges, respectively, of window 714 of elongate body 702. The window may accommodate both soft and hard tissue when, the device is forcibly applied to the surface of a target tissue site. In. some embodiments, the blades may include the angled edges, which facilitate shearing of target tissue. In alternative embodiments, the blades may have any of a number of alternative shapes and configurations. In some embodiments, the distal portion of body 702 may have a very low profile (height compared to width), as shown in side view FIGS. 6 A through 8B, where only the blades protrude from the top surface of the elongate body. In some embodiments, the lower surface of elongate body is an example of a protective or non-tissue-modifying surface 712.

In one embodiment, as shown in FIGS. 9A and 10A, proximal blade 910 may be coupled with a push mechanism, such as the multi-wire drive mechanism 918 (see also reference 718 in FIGS. 8A and 8B). Drive mechanism 918 may be coupled to and translated by actuator 706 on handle 704 and may be used to drive or push proximal blade 710 distally to contact the cutting edge of distal blade 708, thus cutting tissue. In one embodiment, as shown in FIGS. 9B and 8B, distal blade 908 may be coupled with a pull mechanism, such as two pull-wires 916 (shown, within a channel or guide). Pull-wires may be coupled to and translated by actuator 706 on handle 704 and may be used to drive or pull distal blade 708 proximaily to contact the cutting edge of proximal blade 710, thus cutting tissue. In some alternative embodiments, the distal blade 708 may be pulled and the proximal blade 710 may be pushed such that each blade moves toward the opposite blade to cut.

Other alternative mechanisms for driving blades, such as gears, ribbons or belts, magnets, electrically powered, shape memory alloy, electromagnetic solenoids and/or the like, coupled, to suitable actuators, may be used in alternative embodiments. As mentioned, in one embodiment distal blade and/or proximal blade may have an outwardly curvilinear shape along its cutting edge. Alternatively, distal blade may have a different blade shape, including flat, rectilinear, v-shaped, and inwardly curvilinear (concave vs. convex). The cutting edge of either blade 110 may have a sharp edge formed by a simple bevel or chamfer. Alternatively or in addition, a cutting edge may have tooth-like elements that interlock with a cutting edge of an opposing blade, or may have corrugated ridges, serrations, rasp-like features, or the like. In various embodiments, both blades 110 may be of equal sharpness, or alternatively one blade 110 may be sharp and the other substantially flat to provide a surface against which the sharp blade 110 may cut, Alternately or in addition, both cutting edges may be equally hard, or a first cutting edge may be harder than a second, the latter of which deflects under force from the first harder edge to facilitate shearing of the target tissue.

In some embodiments, all or a portion of elongate body, such as the lower surface 712, may include a lubricious surface for facilitating manipulation of the tool in the surgical space and at the anatomical site. The lubricious lower surface also provides a barrier between blades and non-target tissue in the surgical space

In some embodiments, when at least one of the blades is moved to cut tissue, at least some of the cut tissue may be captured in a hollow interior portion of elongate body. Various embodiments may further include a cover, a cut. tissue housing portion and/or the like for collecting cut tissue and/or other tissue debris. Such collected tissue and debris may then be removed from the patient during or after a tissue modification procedure. During a given tissue modification procedure, distal blade, for example, may be drawn proximally to cut tissue. allowed to retract distally, and drawn proximally again to further cut tissue as many times as desired to achieve a desired amount of tissue cutting.

The blades may be made from any suitable metal, polymer, ceramic, or combination thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for the blades or for portions or coatings of the blades may be chosen for their electrically conductive or thermally resistive properties. Suitable polymers include but are not limited to nylon, polyester, Dacron®, polyethylene, acetal. Deirin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In various embodiments, blades may be manufactured using metal injection molding (MIM), CMC machining, injection molding grinding and/or the like, Pull wires or drive mechanisms may be made from metal or polymer and may have circular, oval, rectangular, square or braided cross-sections.

Depending on the tissue to be treated or modified, activating blades (or other tissue modifying members in alternative embodiments) may cause them to modify target tissue along an area having any of a number of suitable lengths. In use, it may also be advantageous to limit the extent of action of blades or other tissue modifying members to a desired length of tissue, thus not allowing blades to affect tissue beyond that length. In so limiting the effect of blades, unwanted modification of, or damage to, surrounding tissues and structures may be limited or even eliminated. In one embodiment, for example, where the tissue modification device is used to modify tissue in a spine, blades may operate along a length of target tissue of no more than 10 cm, and preferably no more than 6 cm, and even more preferably no more than 3 cm. Of course, in other parts of the body and to address other tissues, different tissue modification devices may be used and tissue modifying members may have many different lengths of activity. In one embodiment, to facilitate proper location of tissue modifying members, such as blades, relative to target tissue, the tissue modifying members and/or the elongate body and/or one or more additional features intended for just such a purpose may be composed, of a material readily identifiable via x-ray, fluoroscopic, magnetic resonance or ultrasound imaging techniques.

In various embodiments, a number of different techniques may be used to prevent blades 110 (or other tissue modifying members) from extending significantly beyond the target tissue. In one embodiment, for example, preventing blades 110 from extending significantly beyond the target tissue involves holding tissue modification device 102 as a whole predominantly stable to prevent device 102 from translating in a direction toward its proximal portion or toward its distal portion while activating blades 110. Holding device 102 stable is achieved by anchoring one end of the device and applying tensioning force at or near the other end, as described further below.

In some embodiments, pull wires may be retracted proximally by squeezing the actuator proximally. In an alternative embodiment, squeezing the actuator may cause both of the blades to translate inward so that they meet approximately in the middle of the window. In a further embodiment, the distal blade may be returned to its starting position by a pulling force generated from the distal end of the device, for example by using a distal actuator that is attached to distal wires, or by pulling on the distal guide member which is attached to the distal blade. In yet another alternative embodiment, the proximal blade may be moved to cut by a pulling force generated from the distal end of device, for example by using a distal actuator that is attached to distal wires, or by pulling on the distal guide member which is attached to proximal blade. In yet another embodiment, squeezing actuator may cause proximal blade to move distally while the distal blade stays fixed. In other alternative embodiments, one or more blades may move side-to-side, one or more blades may pop, slide or bow up out of the window when activated, or one or more blades may expand through window. In another embodiment, one or more blades and/or other tissue modifying members of device may be powered devices configured to cut, shave, grind, abrade and/or resect target tissue. In other embodiments, one or more blades may be coupled with an energy transmission device, such as a radiofrequency (RF) or thermal resistive device, to provide energy to blade(s) for cutting, ablating, shrinking, dissecting, coagulating or heating and thus enhancing tissue modification. In another embodiment, a rasp or file may be used in conjunction with or coupled with one or more blades. In any of these embodiments, use of actuator and one or more moving blades provides for tissue modification with relatively little overall translation or other movement of tissue modification device. Thus, target tissue may be modified without extending blades or other tissue modification members significantly beyond an area of target tissue to be treated.

In some embodiments, the device described herein may be disposable. In some embodiments, the device described herein may include a neural localization element. In some embodiments, the neural localization element may be at least one electrode configured to emit stimulation from at least one side (e.g. the cutting side) of the device. The stimulation element may be coupled to one of the blades and/or to the elongate body. Alternatively, it may be coupled to the distal tip of the device. In some embodiments, a threshold stimulation amount may elicit an EMG response in the patient, and depending on the magnitude of the stimulation amount, the location of the nerve with respect to the device may be determined. Alternatively, in some embodiments, the neural localization element may include a visualization element such as a camera, endoscope, or microscope. For example, the device may include at least one fiber optic bundle, CCD image sensor, or CMOS image sensor, or any combination thereof. In some embodiments the visualization element may be positioned on the distal tip of the device. Alternatively, the visualization element may be positioned such that it may visualize the window between the cutting blades. The visualization element may be coupled to one of the blades and/or to the elongate body.

As described above, the device may further include a tissue capture region or mechanism configured to capture and/or store tissue that has been cut and/or modified by the device. For example, a portion of the shaft coupling the cutting blades to the proximal handle may include a chamber that receives, collects, and stores tissue.

In some embodiments, the device may further include suction and/or irrigation capabilities. Suction and/or irrigation may aid in visualization, tissue capture, tissue modification, and/or tissue release from the device or into the storage region, bleeding management, and/or any other suitable function. In one example, the suction and/or irrigation capabilities may run from the distal tip, through the proximal handle, and include connection port(s) sized and configured to couple to standard suction and/or irrigation sources,

Any of the procedures described herein can be done in combination with other techniques including an open or minimally invasive decompression procedure where tools such as Rongeurs and powered drills are used to remove tissue primarily around the proximal end of nerve root (lateral recess). Such techniques may include laminotomies, etc.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for modifying tissue, the device comprising:
a rigid elongate body having a thin distal portion that is bent relative to the elongate body, wherein the distal portion comprises a cutting window and the distal portion is slightly wider than the elongate body;
a bent region between the elongate body and the distal portion, wherein the bent region is rigid;
a movable blade disposed along one side of the cutting window and configured to extend across the cutting window to cut tissue; and
a handle at the proximal end of the body, wherein the handle includes an actuator configured to move the movable blade towards a second blade;
wherein the distal portion has a height, a length and a width, and wherein the height is less than the width, further wherein the height is not more than 10 mm at any point along its length, and the width is not more than 20 mm at any point along its length.

2. The device of claim 1, wherein the bent region comprises an invagination configured to stiffen the bent region.

3. The device of claim 1, wherein the distal portion has a height of not more than 5 mm at any point along its length and a width of not more than 10 mm at any point along its length.

4. The device of claim 1, wherein the distal portion has a height of not more than 2 mm at any point along its length and a width of not more than 5 mm at any point along its length.

5. The device of claim 1, wherein the second blade is configured to move across the cutting window towards the movable blade when the movable blade extends across the cutting window.

6. The device of claim 1, wherein the movable blade has an angled cutting edge.

7. The device of claim 1, wherein the movable blade and the second blade have angled cutting edges.

8. The device of claim 1, wherein the actuator is configured to pull the second blade toward the first blade.

9. The device of claim 1, wherein the actuator is configured to push the first blade toward the second blade.

10. The device of claim 1, wherein a distal portion is curved.

11. The device of claim 1, wherein the bend region forms an angle of between 180 degrees and 90 degrees between the elongate body and the distal portion.

12. The device of claim 1, further wherein the actuator connects to a pull wire that moves the movable blade towards the second blade.

13. The device of claim 1, further comprising a guidewire coupler at the distal portion of the device.

14. The device of claim 1, further comprising a guidewire lumen at the distal portion of the device configured to pass a guidewire.

15. A device for modifying tissue, the device comprising:
a rigid elongate body having a thin distal portion that is bent at a fixed angle between about 180 and 90 degrees relative to the elongate body, wherein the distal portion comprises a cutting window, and wherein the distal portion is slightly wider than the elongate body;
a rigid bent region forming the fixed angle between the elongate body and the distal portion;
a movable blade disposed along one side of the cutting window and configured to extend across the cutting window to cut tissue; and
a handle at the proximal end of the body, wherein the handle includes an actuator configured to move the movable blade towards a second blade;
wherein the distal portion has a height, a length and a width, and wherein the height is less than the width, further wherein the height is not more than 5 mm at any point along its length, and the width is not more than 10 mm at any point along its length.

16. The device of claim 15, wherein the bent region comprises an invagination configured to stiffen the bent region.

17. The device of claim 15, wherein the distal portion has a height of not more than 2 mm at any point along its length and a width of not more than 5 mm at any point along its length.

18. The device of claim 15, wherein the second blade is configured to move across the cutting window towards the movable blade when the movable blade extends across the cutting window.

19. The device of claim 15, wherein the movable blade has an angled cutting edge.

20. A device for modifying tissue, the device comprising:
a rigid elongate body having a thin distal portion that is bent at a fixed angle between about 180 and 90 degrees relative to the elongate body, wherein the distal portion comprises a cutting window, and wherein the distal portion is slightly wider than the elongate body;
a rigid, invaginated, bent region forming the fixed angle between the elongate body and the distal portion;
a movable blade disposed along one side of the cutting window and configured to extend across the cutting window to cut tissue; and
a handle at the proximal end of the body, wherein the handle includes an actuator configured to move the movable blade towards a second blade;
wherein the distal portion has a height, a length and a width, and wherein the height is less than the width, further wherein the height is not more than 5 mm at any point along its length, and the width is not more than 10 mm at any point along its length.
